(19) United States
(12) Patent Application Publication
(10) Pub. No.: US 2010/0234966 A1
(43) Pub. Date: Sep. 16, 2010

(54) BONE REPAIR OR AUGMENTATION DEVICE

(75) Inventor: Wei Jen Lo, Nottingham (GB)

Correspondence Address: BIRCH STEWART KOLASCH & BIRCH, PO BOX 747, FALLS CHURCH, VA, 22040-0747, US

(73) Assignee: Orthogem Limited, Nottingham (GB)

(21) Appl. No.: 12/301,995

(22) PCT Filed: May 24, 2007
(86) PCT No.: PCT/GB07/01938

§ 371 Date: Jan. 20, 2009

(30) Foreign Application Priority Data
May 24, 2006 (GB) 0610333.7

(51) Int. Cl. A61F 2/28; A61K 35/12; A61K 38/18; A61K 38/43; A61P 19/00; A61F 2/00; B29C 43/02

(52) U.S. Cl. 623/23.51; 424/93.7; 514/12; 424/94.1; 424/400; 264/337; 264/319; 264/332; 264/42

(57) ABSTRACT

Bone repair or augmentation devices comprising a porous body (2) comprising pores (4), the porous body comprising a portion with a reinforcing agent within the pores of that portion of the porous body (8). Methods of making bone augmentation or repair devices are also provided, the devices having a perimeter and an internal region, the method comprising: (a) mixing a biodegradable reinforcing agent with a biomaterial selected from the group comprising ceramic materials and bioactive glasses to form a mixture; (b) heating the mixture to above the softening point of the reinforcing agent; (c) moulding the mixture around at least a portion of the internal region. A further method is a device having a depth, width, a perimeter and internal region, the method comprising: (i) forming a porous body, the porous body comprising a ceramic material having a plurality of pores (ii) placing a mask on at least the upper surface of the porous body to cover some of the pores and to leave some of the pores exposed (iii) at least partially filling the exposed pores with a reinforcing agent and (iv) removing the mask from at least the upper surface of the porous body to expose pores located under the mask.

The present invention is directed towards a bone repair or augmentation device. The device may be used in orthopaedic surgery, including vertebra repair, musculoskeletal reconstruction, fracture repair, hip and knee reconstruction, osseous augmentation procedures and oral/maxillofacial surgery. More particularly, the device may be used for replacement of at least a part of intervertebra discs or a part of a vertebra.

Bones are major weight-bearing and protective parts of human and animal bodies. They can be damaged by breaking, by general wearing out or through disease or infection. It can be desirable to replace damaged regions of bone with healthy bone or bone-substitute.

The spine (i.e. vertebral column) is a flexible bony column extending from the base of the skull to the small of the back. It encloses and protects the spinal cord, articulates with the skull (at the atlas), ribs (at the thoracic vertebrae), and hip girdle (at the sacrum) and provides attachment for the muscles of the back. The spine is made up of individual bones, called vertebra, connected by discs of fibrocartilage and bound together by ligaments. A human adult's vertebral column contains 26 bones whereas a human baby's vertebral column contains 33 bones.

Spinal problems and/or injuries can arise in many different ways. For example, vertebrae may be fractured through result of an accident; intervertebral discs may wear out due to age, accident and/or infection; subjects may be born with spinal defects. Such problems and/or injuries may be treated by replacement of at least a part of a vertebra, replacement of an intervertebral disc or by fusing adjacent vertebrae together.

Currently, in order to fuse adjacent bones (such as vertebrae) for example to reduce pain associated with the adjacent bones moving relative to each other, two separate surgical processes are required. A first surgical procedure is performed to obtain autograft material (i.e. healthy bone) from a patient, e.g. from the pelvic bone. The autograft material is then ground into ‘chips’ and inserted into a ‘cage’. The cage may be metal (such as titanium) or a plastic material (such as PEEK). A second surgical procedure is performed to remove damaged intervertebral disc from the intervertebral space and to insert the filled cage into the intervertebral space. The requirement for two surgical procedures increases the risk of infection, increases the time taken to perform the complete bone replacement and increases the cost of performing the fusing the adjacent bones.

Disadvantages associated with the use of cages, such as titanium cages, include the cage causing problems during x-ray since the cage is not transparent to x-rays. A consequence of a bone bearing weight is that the bone strengthens. Absence of weight-bearing by a bone causes the bone to weaken. Since the, titanium, cage does not biodegrade, the cage (rather than the bone) bears the weight and consequently the bone weakens.

What is required is an improved bone repair or augmentation device that reduces the amount of surgery required, whilst still having sufficient strength to be used effectively.

Synthetic materials for bone grafts are usually made of calcium phosphate ceramics and have a porous structure similar to that of cancellous bone. Many synthetic materials are derived from animals or marine life, such as from bovine bone or coral. These are intended to offer an interconnected macroporous structure and provide intensive osteoconductivity to regenerate and heal the host bone tissue. However, many of these have problems because their precise composition and structure cannot be controlled.

Such synthetic bone grafts typically come with interconnected “macropores”, typically of 100-500 μm diameter. These provide a framework for the host bone to regenerate whilst reducing healing time. The pores allow bone tissue to grow into the bone graft. According to in vitro and in vivo experiments, the host's own bone tissue uses the macroporous structure to grow into the bone replacement material, the material being slowly degraded and being replaced by new bone growth. Ideally, biomaterials used for bone grafts should be microporous with a pore diameter of 1-10 μm. Such micropores have been found to improve the ability of osteoblasts and other cells from the host to bind to the synthetic biomaterial and to allow access of the cells to dissolve the sintered connections between the individual ceramic particles.

Typical commercially available synthetic bone grafts usually have a random distribution of pore sizes and no observable preferred orientation of the interconnected porous structure. Furthermore, they have little or no microporous structure.

For example, U.S. Pat. No. 6,511,510 discloses an osteoinductive biomaterial that is made from calcium phosphate or a glass ceramic. The material is stated to comprise micropores and macropores, the macropores preferably being interconnected. The micropores are only present on the surface of the material. The osteoinductive biomaterial is obtained by sintering a ceramic material. The material is preferably ground with sandpaper to remove chemical surface impurities and the material is then treated with an aqueous solution of an acid. The acid etches the surface of the material, especially the annealed particles' grains boundaries, to produce the micropores. Macropores may be formed using pore-forming agents such as hydrogen peroxide, baking powder or bicarbonate. Negative replica-forming agents such as wax or fiber are also disclosed which will not generate gas in the same way as hydrogen peroxide or baking powder, but will be burned to leave the same shape or pore as the original wax or fiber.

U.S. Pat. No. 6,479,418 discloses a method of preparing a porous ceramic body by mixing a slurry of a ceramic material with a viscous organic phase to obtain a dough, drying the dough and removing the organic phase by thermal decomposition. Foaming agents, such as sodium bicarbonate and citric acid may be used to create “macropores”. The surface of the ceramic body, including the surface of the pores, is stated to have a microporous surface. This is shown in the document as being irregular depressions in the surface of the material surrounded by irregular clumps of fused ceramic particles.

Ceramic materials used to mould natural objects are disclosed in U.S. Pat. No. 5,705,118. The ceramic uses gluten and/or a number of other materials as a binder. This is mixed together as a batch with water or other liquid, prior to spraying or applying onto an object to produce a mould. This is fired to produce a porous body.

The Applicants developed an alternative method of producing artificial bone which allowed the controlled formation of macropores, including the diameter and orientation of the macropores. This was published as WO 02/11781. The method used in that application prepared a mixture of finely divided bio-compatible ceramic powder, an organic binder and a pore-forming agent in an inert liquid to form a body, causing at least some of the macropores to align along a common axis, prior to heating to fix the porous structure and further heating to eliminate residues of the organic binder and pore-forming agent, and to fuse it. This method was shown to produce a series of tube-like macroporous structures. However, the inventors have found that the method used in WO 02/11781 does not allow the size and distribution of micropores to be controlled. Using the method of WO 02/11781 results in the clumping of ceramic particles and an uneven distribution of any micropores is formed.

They were then able to identify a method of producing a biomaterial having a plurality of connecting micropores which are substantially evenly distributed through the entire cross-section of the ceramic material. This improves the ability of a recipient's cells to bind to the biomaterial and integrate it with the recipient's own bone or other tissue.

WO2004/101013 discloses improved porous biomaterials comprising a variety of pore sizes.

One such porous ceramic material is useful for bone repair. However, there is a need to improve the strength or physical resistance of the material to allow it to be more successfully used in positions where the bone replacement or repair material is likely to be knocked and damaged or where extra compressible strength is required.

A first object of the invention is to provide a bone repair or augmentation device comprising a porous body, the porous body comprising pores characterised in that a portion of the porous body additionally comprises a reinforcing agent within the pores of that portion of the porous body. The porous body may comprise a porous biomaterial.

The term biomaterial includes biologically compatible material which preferably is capable of being at least partially resorbed in vivo.

Preferably the reinforcing agent is selected from the group comprising polymers and metals. Preferably the reinforcing agent is biodegradable. The reinforcing agent and/or the material forming the porous body may be absorbed or degraded over a period of time within the body. The period of time may be several months or years during which it may be replaced by new bone growth.

This leaves a portion of the porous body into which new bone may grow to fuse the material to surrounding bone, whilst also providing a reinforced part of the body.

Preferably at least 5%, 10%, 20%, 30%, 40%, 50%, 60%, 70% or 80% of the porous body, but preferably less than 90% of the body contains reinforcing agent.

Preferably, the porous body comprises a plurality of interconnecting macropores which are substantially aligned along an axis. The applicants have previously developed a method of producing artificial bone which allows the controlled formation of macropores, including the diameter and orientation of the macropores. This method was published in international patent application WO 02/11781. The method used in that application prepared a mixture of finely divided bio-compatible ceramic powder, an organic binder and a pore-forming agent in an inert liquid to form a body, causing at least some of the macropores to align along a common axis, prior to heating to fix the porous structure and further heating to eliminate residues of the organic binder and pore-forming agent, and to fuse it. This method was shown to produce a series of tube-like macroporous structures. However, the inventors have found that the method used in WO 02/11781 does not allow the size and distribution of micropores to be controlled as well as possible. Using the method of WO 02/11781 results in the clumping of ceramic particles and an uneven distribution of any micropores is formed.

Alternatively, the use of fibres to form the elongated micropores is also known in the art. The fibres are decomposed on sintering the ceramic material to leave an elongated pore.

The inventors have previously identified an improved method of producing a biomaterial having a plurality of connecting micropores which are substantially evenly distributed through the entire cross-section of the ceramic material. This improves the ability of a recipient's cells to bind to the biomaterial and integrate it with the recipient's own bone or other tissue. This method was published in international patent application WO 04/101013. The general method shown in WO 02/11781 used to align the macropores may be combined with the method of WO 04/101013 to produce improved material with fewer clumps of material. WO 02/11781 and WO 04/101013 are incorporated herein by reference.

The bone repair or augmentation device of the present invention preferably comprises an upper surface and a lower surface and one or more outer edges connecting the upper surface to the lower surface, wherein more reinforcing agent is located in pores towards the outer edges then in pores located in the centre of the device. Alternatively, more reinforcing agent may be located in pores located in the centre of the device than in the pores located towards the outer edges of the device. As a further alternative, two or more reinforcing agents (or mixtures of reinforcing agents) may be used wherein the volume and/or type of reinforcing agent located in the pores results in those pores being reinforced to a greater or lesser extent that other pores which are either not filled with any reinforcing agent or are filled with a different type of reinforcing agent (or mixture of reinforcing agents) and/or filled to a different extent.

In a preferred embodiment, the plurality of interconnecting pores align substantially along an axis running through the device from the upper surface to the lower surface.

Preferably the porous body comprises a biomaterial having a plurality of connecting micropores of an average diameter of between 1 μm and 10 μm substantially evenly distributed through the biomaterial.

That is, the micropores are not confined to the surface of the biomaterial but are found substantially throughout a cross-section through the ceramic material.

Preferably, the average diameter of the micropores is between 2-8 μm, most preferably 5-6 μm.

The micropores may be irregular in shape. Accordingly, the diameter of the micropores, and indeed the macropores and midipores referred to below, are determined by adding the widest diameter of the pore to the narrowest diameter of the pore and dividing by 2.

Preferably, the ceramic material is evenly distributed through the cross-section, that is substantially without clumps of ceramic material forming.

Preferably, the biomaterial comprises a plurality of ceramic particles, each particle being partially fused to one or more adjacent ceramic particles to form a lattice defining the micropores.

Preferably, the biomaterial contains particles having an average particle diameter of 1-10 μm, more preferably at least 2 μm or 4 μm and/or less than 10 μm or less than 6 μm, most preferably 5-6 μm. This particle size range has been found to allow the controlled formation of the micropores.

The average porosity of the biomaterial is preferably at least 50%, more preferably greater than 60%, most preferably between 70-75% average porosity.

Preferably, the biomaterial without reinforcing agent has a compressive strength of at least 1.0 MPa to preferably 10 MPa, more preferably 1.5 MPa, 2 MPa, 3 MPa, 4 MPa, 5 MPa, most preferably between 6 MPa and 7 MPa. Compressive strength may be detected using techniques known in the art. Typically 1 cm³ of sample is compressed during a test.

The inventors have been able to produce biomaterials having reduced wall thicknesses between each macropore. This improves the ability of the biomaterial to be incorporated into the host. Accordingly, preferably the average thickness of ceramic material between each macropore is 20-200 μm, most preferably 50-150 μm, more preferably 50-100 μm.

Preferably the product is bread-like in cross-section with macropores and micropores.

The biomaterial may additionally comprise a plurality of elongated macropores having an average diameter of between 150-500 μm, more preferably 200-400 μm. That is, they preferably have a substantially circular cross-section, and are tube-like. These macropores may have an average length of between 300-3000 μm, more preferably at least 300 μm, at least 400 μm or at least 500 μm and/or less than 3000 μm, less than 2000 μm, less than 1000 μm, or less than 800 μm, most preferably 500-1000 μm. At least a portion of the macropores are preferably interconnecting.

The biomaterial may additionally comprise a plurality of midipores within walls that are formed between the macropores. Midipores are substantially spherical pores which are typically approximately 5-150 μm, especially 50-100 μm or 60-100 μm in diameter. They substantially increase the total porosity without compromising the mechanical strength of the materials. Furthermore, the midipores can be beneficently used to accommodate osteocyte formation, deliver drugs, cell growth factors or other biologically active agents.

The macropores and midipores are preferably themselves interconnected via a plurality of micropores. That is, the macropores, and where present midipores, may be in fluid connection with each other via micropores, instead of or in addition to the interconnected macropores.

The biomaterial may be non-biodegradable or, preferably, biodegradable. The term non-biodegradable includes the inability of the device to be resorbed in vivo. The term biodegradable includes the ability of the device to be partially or fully resorbed in vivo. The device may be completely biodegradable. That is, over time the device may be completely resorbed in vivo. Preferably the biodegradation characteristics of the biomaterial is such that the bone augmentation or replacement device is weight bearing for at least 6 months, 12 months, 18 months, 24 months, 30 months, 36 months, 48 months, 60 months. Most preferably, the device is weight bearing for around 24 months.

The reinforcing agent is preferably provided within the elongated macropores, and preferably additionally the midipores, where present. However, depending on, for example, the particle size or viscosity of the reinforcing agent, it may also be present within the micropores of the porous body.

Preferably the device comprises a biomaterial selected from the groups comprising:
(i) ceramics and
(ii) bioactive glasses

The ceramic material used may be any non-toxic ceramic known in the art, such as calcium phosphate and glass ceramics. Preferably the ceramic is not a silicate. Most preferably the ceramic material is a calcium phosphate, especially α- or β-tricalcium phosphate or hydroxyapatite, or mixtures thereof. Most preferably, the mixture is hydroxyapatite and β-tricalcium phosphate, especially more than 50% w/w hydroxyapatite, most preferably 70% hydroxyapatite and 30% β-tricalcium phosphate.

Preferably the bioactive glass comprises a controlled release glass. Preferably the bioactive glass comprises a network former other than SiO₂. Non-SiO₂. network formers are preferred for the reasons reviewed in Griffon, D. (Academic Dissertation entitled Evaluation of Osteoproductive Biomaterials: Allograft, Bone Inducing Agent, Bioactive Glass and Ceramics; University of Helsinki, Finland (2002)). This is because bioactive glasses containing SiO₂ as a network former results in slow and incomplete resorption. Preferably the bioactive glass comprises a controlled release glass (CRG). CRGs are inorganic polymers based on phosphates of sodium and calcium converted into a glassy form. CRGs do not contain SiO₂. When exposed to tissue fluids, traditional bioactive glasses form a bonding layer of biological hydroxy-carbonate-apatite with an underlying layer of silica gel, while CRGs dissolve completely in water and create an acidic environment.

Another preferred bioactive glass is Wollastonite.

Preferably the non-silicate network former is P₂O₅. Preferably P₂O₅ is present in the glass at 42-49 mole %. Preferably the remainder of the glass comprises 10-40% mole % CaO and Na₂O.

Preferred silicate free glasses include those available from Giltech Ltd, Ayr, UK under the trade mark Corglaes.

Preferably the reinforcing polymer is a thermoplastic. Alternatively, the reinforcing polymer may be a thermosetting plastic. Preferably, the polymer is selected from the group comprising: polycaprolactone (PCL), polyesters, polyetheretherketon (PEEK), polyphosphazenes, polyacetals, polyalkanoates, polyurethanes, poly (lactic acid) (PLA), poly (L-lactic acid) (PLLA), poly (DL-lactic acid), poly-DL-lactide-co-glycolide (PDLGA), poly (L-lactide-co-glycolide) (PLLGA), polyorthoesters, polycarbonates, ABA tri-block co-polymers with A blocks of semicrystalline polyglycolic acid (PGA) and a B block of amorphous trimethylene carbonate (TMC), also known as polyglyconates, polyhydroxyalkanoate, polybutylene succinate (PBS), aliphatic-aromatic copolyesters, polybutylene adipate/terephalate, polyhydroxybutyrate, polyhydroxyvalerate, polybutylene succinate adipate, polyethylene terephthalate (PET), polymethylene adipate/terephthalate, polyhydroxyhexanoate and poly(d,l-lactide-co-glycolide). Erodible polymers are particularly preferred. Suitable erodible polymers include: polydioxanone, poly(ε-caprolactone), polyanhydride, poly(ortho ester), co-poly(ether-ester), polyamide, polylactone, poly(propylene fumarate) (H[—O—CH(CH₃)—CH₂—O—CO—CH═CH—CO—]ₙOH), poly(lactic acid), poly(glycolycic acid), poly(lactide-co-glycolide) and combinations thereof. Suitable naturally produced polyesters include poly-hydroxybutyrate-co-polyhydroxyhexanoates (PHBHs) resins. The PHBH resin is derived from carbon sources such as sucrose, fatty acids or molasses via a fermentation process. These are aliphatic-aliphatic co-polyesters. PHBH polyesters are available under the Nodax' trade mark, developed by Kaneka Corp. and marketed by Proctor & Gamble Co.

PCL are especially preferred as the strength provided by the PCL and the biodegradability is especially suitable. PCL has a relatively slow degradation rate, i.e. in vivo it takes around 2 years to degrade. PCL is also very amenable to moulding accurately and to reproduce reliably devices and therefore is very amenable to use in the present invention.

PLA polyesters are ‘renewable resource’ polyesters and are commercially available, for example: Lacea™ (Mitsui Toatsu, Japan) and NatureWorks' (Cargill Dow, USA). PCL polyesters are ‘synthetic aliphatic’ polyesters and are commercially available, for example, Tone™ (Union Carbide, USA), CAPA™, (Solvay, Belgium), Placeel™ (Daicel Chemical Indus. Japan). PBS polyesters are also ‘synthetic aliphatic’ polyesters and are commercially available, for example, Bionelle™ (Show Highpolymer, Japan) and SkyGreen BDP™ (SK Polymers, Korea). Aliphatic-aormatic copolyesters (AACs) combine the biodegradable properties of aliphatic polyesters with the strength and performance of aromatic polyesters. AACs may be blended with TPS (ThermoPlastic Starch) to reduce costs. AAC plastics are commercially available, e.g. Ecoflex™ (BASF) and Eastar Bio™ (Eastman). The AACs available under these trade names are provided at a number of specific grades, each suitable for a particular application. Some modified polyethylene terephalates (PETs) are susceptible to biodegradation. Biodegradable PETs include PBAT (polybutylene adipate/terephthalate) and PTMAT (polytetramethylene adipate/terephthalate). Biodegradable PETs are commercially available, e.g. Biomax™ (DuPont).

Particularly preferred polymers include PCLC (Poly(f-caprolactone)-montmorillonite), PLA, PGA and PLGA. PCLC has been approved by the United States Food and Drug Administration (FDA) for use in sutures. PLA, PGA and PLGA have been approved by the FDA for use as replacement bone material.

Preferably the reinforcing metal is magnesium or a magnesium alloy. Preferably the magnesium alloy comprises at least one metal selected from the group comprising: aluminium, cadmium, cerium, dysprosium, lanthanum, lithium, manganese, neodynium, prascodynium, silicon, silver, ytrium, zinc and zirconium. Alternatively, the reinforcing agent may be selected from the group comprising titanium, titanium alloys, cobalt alloys and stainless steel.

Optionally, the reinforcing metal is degradable within the body of a patient. The reinforcing metal is most preferably magnesium or a magnesium alloy.

Staiger, M. P. et al (2006) (Biomaterials: Vol 27(9):1728-34) have reviewed the use of magnesium and its alloys in orthopaedic biomaterials. Advantages of magnesium and magnesium alloys include low density; high fracture toughness; elastic modulus and compressive yield strength are more similar to those of natural bone than is the case for other commonly used metal implants; magnesium is essential to human metabolism and is naturally found in bone tissue. Magnesium, and its alloys, has low corrosion resistance, especially in electrolytic, aqueous environments, and consequently may corrode in vivo—this in vivo corrosion forms a soluble non-toxic oxide that is harmlessly excreted in urine. Furthermore, these materials may have stimulatory effects on the growth of new bone tissue.

The reinforcing agent may comprise one or more polymers and/or one or more metals. The polymer(s) and/or metals strengthen the porous material.

Preferably the degradation characteristics of the device are such that it is load-bearing for at least 6 months, 12 months, 18 months, 24 months, 30 months, 36 months, 48 months, 60 months. Most preferably, the device is weight bearing for around 24 months.

The reinforcing agent and/or the device may be coated with a coating to obtain a product with a desired degradation characteristics.

A further preferred feature is that the portion of the device not comprising reinforcing agent, for example the internal region, comprises one or more biologically or pharmaceutically active compounds. These may be incorporated into the pores and in use may be used to stimulate cell growth around and into the biomaterial. For example, stem cells may be incorporated into the pores. The stem cells may be adult stems cells. Preferably the stem cells are mesenchymal stem cells. Alternatively, or in addition, growth factors, such as transforming growth factor (TGF-131), one or more bone morphogenetic proteins (for example one of more of BMP-1, BMP-2, BMP-3, BMP-4, BMP-5, BMP-6, BMP-7, BMP-8A, BMP-8B, BMP-9, BMP-10, and/or BMP-11, most preferably BMP-2 and/or BMP-7) or a precursor thereof or osteogenic protein (0P-1) may be incorporated into the biomaterial. Alternatively, or in addition, one or more other osteoinductive and/or osteogenic agents may be incorporated into the biomaterial. An advantage of using stem cells in combination with a growth factor, such as a BMP, is that the growth factor may stimulate the stem cells to differentiate into osteoblasts and therefore enhance bone growth and/or enhance absorption of the graft by the bone. Further materials such as enzymes, vitamins (including Vitamin D) and trace minerals/materials such as zinc (for example in the form of a salt) may also be incorporated. The reinforcing agent may be non-biodegradable or, preferably, biodegradable. The term non-biodegradable includes the inability of the device to be resorbed in vivo. The term biodegradable includes the ability of the reinforcing agent to be partially or fully resorbed in vivo. The reinforcing agent may be completely biodegradable. That is, over time the reinforcing agent may be completely resorbed in vivo. Preferably the biodegradation characteristics of the reinforcing agent is such that the bone augmentation or replacement device is weight bearing for at least 6 months, 12 months, 18 months, 24 months, 30 months, 36 months, 48 months, 60 months. Most preferably, the device is weight bearing for around 24 months.

In a preferred embodiment, the central region of the device resorbs faster than the peripheral region of the device. Alternatively, the peripheral region of the device resorbs faster than the central region of the device. As a further alternative, the peripheral and central regions of the device resorb at substantially the same speed. The terms central and peripheral particularly apply to a central cylindrical core surrounded by a tube-like peripheral region. However, these terms also apply to non-cylindrical structures.

In a preferred embodiment, the device further comprises at least one aperture through which a surgical screw may be inserted. This assists fixing of the device to a bone. Preferably the device comprises at least 2, 3, 4, 5, 6, 7, 8, 9, 10 apertures through which a surgical screw may be inserted. Preferably the apertures comprise a thread for insertion of a threaded screw. Preferably at least one of the apertures is formed in the periphery of the device. Most preferably, all of the apertures are formed in the periphery of the device. Preferably the apertures are formed after the porous body is formed and/or after the pores of the porous body has been at least partially filled with reinforcing agent. Alternatively, the apertures may be formed during formation of the porous body or during the at least partial filling of the pores of the porous body.

The device of the present invention is particularly suited to at least partial replacement of an intervertebral disc. The device may be shaped such that it is suitable for full replacement of an intervertebral disc. Such shaping may be carried out manually or by machine, for example by moulding.

The device of the present invention has many advantages over previously known devices. Previously, a surgeon needed to take an autograft from a bone (such as a pelvis) of a patient, grind the bone to small granules, pack the granules into the centre of a spinal cage, insert the packed cage into a site of damage (e.g. into an intervertebral space to replace an intervertebral disc) of the patient and screw the packed cage into place. This requires two surgical procedures, each of which involves the danger of exposing a patient to potential infection and other risks associated with invasive surgery. The advantage of the present invention is that it removes the requirement of the first surgical procedure, i.e. taking an autograft.

A second object of the invention is to provide a method for making a bone repair or augmentation device, the device having a perimeter and an internal region.

The method may comprise:

(a) mixing a biodegradable reinforcing agent with a ceramic material to form a mixture;
(b) heating the mixture to above the softening point of the reinforcing agent;
(c) moulding the mixture around at least a portion of the ceramic material.

As an alternative, or in addition, to the ceramic material, the reinforcing agent may be mixed with another biomaterial, such as a bioactive glass, to form the mixture. Ceramics and bioactive glasses are described throughout this document.

Preferably the reinforcing agent is a polymer or metal as described above.

Preferably the moulding of step (c) comprises forming the product of (b) into an elongate shape.

Preferably the moulding of step (c) comprises wrapping the elongate shape around the exterior of the internal region, for example around the exterior of a body of bio material, to form a perimeter.

Preferably the product of the method is allowed to cool prior eventual use in a surgical procedure.

Optionally, prior to the wrapping step described above, the elongate shape may be further softened for example by dipping it into a hot liquid, preferably a boiling liquid, or exposing it to vapour. Preferably the liquid is water.

For a device comprising a metal reinforcing agent, the method of production of the perimeter may differ slightly. For example, the reinforcing agent may be provided in powder form. Powder metallurgy methods may be used.

Preferably the metal is mixed with a biomaterial. Preferably the biomaterial is a ceramic. Preferably the mixture of metal and biomaterial is pressed into a mould. Preferably the pressing initially forms a loosely-packed body. Preferably the loosely-packed body ‘holds’ its shape and therefore is suitable for forming a perimeter of the device. The loosely-packed body may comprise a cage.

Further pressing and/or heat treatment may be used to further press the loosely-packed body into a compact body. The further pressing may be carried out prior to or after locating at least a portion of an internal region, for example a ceramic insert, adjacent the perimeter to form a composite device. The perimeter and internal region may be held together by a ‘push-fit’. Alternatively, or in addition, in order to fix the internal region to the perimeter, the composite device may be pressed as described above. Alternatively, or in addition, the components of the perimeter and/or the components of the composite device may be fused together using any method known in the art. One suitable method of fusing is annealing. Annealing may be carried out in a furnace, such as a vacuum furnace or an argon or nitrogen filled furnace.

Preferably the fusing does not comprise oxidation of the metal part of the device. Preferably the fusing temperature is from 200 to 600° C. The fusing temperature may be varied to obtain a perimeter with a desired mechanical strength.

Optionally, the product may be pressed. Preferably the pressing is by an automated press. Preferably the pressing is in a vertical direction, that is preferably the pressing compresses the depth of the perimeter. Preferably the press compresses the perimeter to a greater extent than it compresses the internal region. Preferably, the press does not compress the internal region. The pressing may cause the perimeter to irreversibly adhere to the internal region.

Optionally the product may be cooled. Preferably the product is cooled prior to eventually using the product in a surgical procedure. Cooling may be carried out by allowing the product to naturally cool to the ambient temperature. Alternatively, cooling may be accelerated by any method known in the art for example by exposing the product to a low temperature gas or liquid

After manufacturing, the package may be cleaned and sterilised, for example, by gamma-radiation or electron beam radiation.

Preferably the internal region of the resultant device has a greater height than the perimeter region of the resultant device.

Preferably the product is sterilised, for example by exposing it to gamma radiation.

Preferably the reinforcing agent is biodegradable. Preferably the reinforcing agent is a polymer or metal as described above.

Preferably the biodegradable polymer is selected from PCL, PLA, PGA and PGLA more preferably the biodegradable polymer is high density PCL. Preferably at least one of the biomaterial of the perimeter or a material making up the internal region is porous. Preferably the internal region comprises a ceramic material. Preferably the pores of the porous material are substantially aligned a common axis. Most preferably the porous material is the ceramic material described in WO02/11781 or WO04/101013.

Preferably the biodegradable polymer is provided in bead form. Preferably the beads are from about 250 μm to about 5 mm in diameter. Preferably the ceramic is provided in granule form. The shapes and sizes of the beads and granules may be altered to alter the characteristics of the device.

Preferably the metal is one of those described above. Preferably the metal is provided in powder form. Preferably the powder has particles from 10 μm to 1000 μm in diameter, more preferably from 100 μm to 500 μM in diameter.

Preferably the mixture of reinforcing agent and biomaterial comprises approximately a 50:50 (by weight) ratio. However, the relative amounts of reinforcing agent and ceramic may be altered in order to alter the characteristics of the device. For example, where the reinforcing agent comprises a polymer such as PCL, PLLA or PGLA, increasing the amount of polymer increases the workability of the device but also decreases the strength of the device.

One of the characteristics of the device that may be altered is the compressive strength in order to allow devices to be made which have suitable compressive strengths for their intended use. For example, a device may mimic the compressive strength of the bone or other material (such as intervertebral disc) that it is intended to replace and/or repair.

Preferably the heating of step (b) is carried using steam, e.g. using a steam oven. Preferably the heating is carried out indirectly, for example the reinforcing agent and ceramic may be contained within a glass vessel.

The elongate shape may have any suitable cross-section, for example circular, square, rectangular. Preferably the thickness of the elongate shape is between 1 mm and 10 mm, more preferably between 2 mm and 5 mm.

Preferably, the ratio of the thickness of the elongate shape to the longest diameter of body of ceramic material is from 1:20 to 3:10. The term ‘diameter’ means a straight line from one edge of a shape to another edge of the shape which lines passes through the geometric centre of the shape. The shape may be regular, such as a circle or square, D-shaped or irregular.

Alternatively, the method for making a bone repair or augmentation device, the device having a perimeter and an internal region may comprise:

(i) forming a porous body, the porous body comprising a ceramic material having a plurality of pores
(ii) placing a mask on at least the upper surface of the porous body to cover some of the pores and to leave some of the pores exposed
(iii) at least partially filling the exposed pores with a reinforcing agent and
(iv) removing the mask from at least the upper surface of the porous body to expose pores located under the mask.

Alternatively, or in addition, the porous body may comprise another biomaterial such as a bioactive glass. Suitable ceramics, bioactive glass and reinforcing agents are described above.

The method may further comprise, at the same time as or after step (ii), placing a mask on the lower surface of the porous body to cover some of the pores and to leave some of the pores exposed and at the same time as or after step (iv) removing the mask from the lower surface of the porous body to expose pores located under the mask.

Preferably the mask is stable at high temperatures. Preferably the mask comprises a rubber material.

The mask may be placed on a surface of the porous body. Alternatively, the mask may be temporarily or permanently fixed to the surface of the porous body. For example, the mask may be fixed to the porous body by a clamp and/or fixed by a suitable adhesive. The clamp should not directly contact the porous body.

The at least partial filling of the exposed pores is by immersing at least a portion of the porous body in a reinforcing agent and/or by injecting a reinforcing agent into the pores.

For a device in which the pores are substantially aligned along an axis running between the upper and lower surface of the device, and in which it is desired to fill pores in one region (such as around the perimeter of the device) but not to fill pores in a another region (such as in the centre of the device) such selective filling may be achieved by masking-off the pores which are not to be filled. Substantial alignment of the pores should prevent flow of reinforcing agent between the pores which are to be filled and the pores which are not to be filled. Pores may be filled by immersion of the porous body in a reinforcing agent and/or by injecting reinforcing agent into the pores.

An alternative method of at least partial filling of pores is to immerse a porous device in a volume of reinforcing agent. The extent to which pores are filled will be proportional to the immersion time period. For example, if full filling of the pores is required, the device will be immersed for a long period of time. If only partial filling of the pores is required, the device will be immersed for a short period of time. The skilled person will readily be able to correlate the length of time required to achieve a desired extent of filling for a given reinforcing agent.

Immersion may be carried out in a vacuum oven. The temperature inside the vacuum oven is altered to be suitable for a given reinforcing agent. This encourages the displacement of air within the pores and replacement by reinforcing agent.

For a substantially spherical device, the pores may be aligned to substantially radiate from the centre of the device to the surface of the device. Therefore, when a porous spherical device is immersed in reinforcing agent for a short period of time, the reinforcing agent will fill only pores towards the surface of the sphere and the reinforcing agent will not penetrate to the pores in the central core of the sphere. In contrast, when the porous spherical device is immersed in reinforcing agent for a long period of time, the reinforcing agent will fill the pores towards the outer surface of the sphere and the pores in the central core of the sphere. This process also applies to devices whose 3D shapes are such that it is desirable to have a central core reinforced to a different extent to the outer regions, for example cubes and non-uniform shapes which are substantially non planar. A pore-forming agent, such as a yeast, may be used to generate a device with suitably aligned pores.

More preferably, the pores are filled with reinforcing agent by injection. Preferably the injection is by using injection moulding. Reinforcing agent is injected through an injection moulding nozzle into the area of the body to be reinforced. An advantage of injection moulding is that filling of the pores at high pressure results in good fusion between the reinforcing agent and the porous body. In addition, injection moulding is relatively easy to control and standardise. Also, injection moulding can be carried out quickly. Therefore, use of injection moulding allows manufacture of large numbers of devices with a high level of batch-to-batch uniformity.

Preferably during the filling process the reinforcing agent is in a fluid or molten state. Alternatively, during the filling process the reinforcing agent may be in a powder or pellet state and following filling the device and reinforcing agent may be heated to turn the reinforcing agent into liquid form. Following heating, the device and reinforcing agent may be allowed to return naturally to room temperature or may be cooled to a desired temperature to gel or to solidify the reinforcing agent. The desired temperature may be room temperature, body temperature (i.e. around 37° C.), the solidifying point of a reinforcing agent, the gelling point of a reinforcing agent or any other desirable temperature.

The method for making a bone repair or augmentation device is particularly suitable for making a device as described above.

The method of WO 04/101013 involves (i) preparing a mixture of finely divided bio compatible ceramic particles with a coating agent; (ii) causing the coating agent to coat the ceramic particles to form coated particles; (iii) causing the coated particles to form a body; and (iv) heating the body to eliminate residues of the coating agent and to partially fuse the ceramic particles, thereby to produce a fused biomaterial.

Coating the particles was found to improve the distribution of the particles through the finely fused product and to produce a substantially uniform product with substantially evenly distributed micropores.

Suitable coating agents include those comprising starch, agar, polyethylene glycol (PEG), hydroquinone, ethyl cellulose or tetrapropylammonium. The starch is preferably provided as corn flour, potato starch or rice powder, most preferably tapioca powder.

Where the coating agent is liquid, for example PEG, simply mixing the ceramic particles in the coating agent may coat the particles. Alternatively, some coating agents, such as the starch and agar coating agents may be mixed with an inert liquid, such as water, in a powder form, and heated to allow the starch or agar to form a polymer coating around the particles. Heating liquids containing starch causes the starch to polymerise and causes it to thicken the liquid in a similar manner to adding corn flour to thicken gravy when cooking.

The inventors found that where the mixture of ceramic particles and coating agent needs to be heated, then it is convenient to mix the components, including where necessary the inert liquid, and then heat the mixture in a steam generator, such as a rice cooker. Heating the mixture in steam allows the mixture to be heated in a controlled manner, whilst allowing the mixture to remain moist. The time will, of course, vary depending on the quantities used. Heating such mixtures of material, typically produces a body having a dough-like consistency. Preferably the mixture is heated to about 100° C. for typically 20-30 minutes.

The body is finally heated to eliminate residues of the coating agent and to partially fuse the ceramic particles to produce a fused biomaterial. This final heating step is also known as an annealing or sintering step and typically uses temperatures of about 1200° C. to about 1450° C., preferably 1200-1350° C. Temperature and duration of heating will depend upon the size of the sample and the initial ceramic concentration and the type of ceramic material used. Furthermore, the temperature is controlled to prevent fusion of the micropores. Typically, the body is annealed for 1 to 2 hours.

Typically the weight ratio between the ceramic powder and the total amount of carbohydrate and gluten powder is between about 1.087:1 to about 1.163:1. The weight ratio of ceramic powder to inert liquid is typically between about 1.042:1 to 1.316:1.

This process, as well as producing the biomaterial of the first aspect of the invention, has been found to reduce the appearance of large voids within the material, thus reducing wastage of biomaterial which would otherwise be disposed of due to the voids.

The ceramic particles may also be mixed, prior to coating, with a dispersing agent. The dispersing agent allows the ceramic powder to be homogeneously mixed with, for example, the inert liquid such as water. Without the dispersing agent, the ceramic particles will separate from the water within minutes. The function of the dispersing agent is to prevent the precipitation of the powder and to allow it to be homogeneously dispersed within the water.

Preferred dispersing agents include acid-based solutions, polymers such as phosphates and acrylate polymers, ammonia, phosphoric acids such as orthophosphoric acid, or an ammonium salt of an acrylate or methacrylate polymer such as ammonium polyacrylate and ammonium polymethacrylate. Relatively small amounts of the dispersing agent need be used, for example for 100 ml of inert liquid only 0.5 ml to 1 ml of dispersing agent may be required.

The body formed from the coated particles may be mixed with an organic binder prior to the final heating step. The organic binder is preferably a carbohydrate powder, such as corn flour or wheat flour. However, the inventor has identified that adding high-gluten flours (also known as strong flours), or indeed extracted gluten, improves formation of the final product. Gluten is the reserve protein of seeds, such as wheat grain. Typically, it contains at least 85% protein and is a mixture of gliadin and glutenin, along with globulin and albumin.

If it is desired to form macropores, then it is necessary to use a pore-forming agent. This agent is allowed to form a pore-forming structure in the body and then is heated to fix the porous structure. This heating step may be at a lower temperature than the final sintering step, typically 100-230, 130-230 or 150-230° C. This is preferably in a humidity-controlled oven, for example in steam. Generally, this stabilisation of the pore-forming structure can be achieved in less than 1 hour, generally 5-50 minutes, for example 15-45 minutes. This will vary depending on the size of the body.

The pore-forming agent may be mixed with the organic binder and the body may be a chemical pore-forming agent such as hydrogen peroxide, disodium diphosphate or sodium bicarbonate. However, most preferably the pore-forming agent is a micro-organism such as a yeast or bacterium. Such micro-organisms preferably form carbon dioxide by metabolising a carbohydrate, such as a sugar which may be added to the organic binder. The advantage of using a micro-organism is that the size of the macropores may be carefully controlled. Furthermore, the pore-forming action of the micro-organism can be easily stopped simply by heating the body to kill the micro-organism.

If yeast is used, then preferably a yeast enhancer is also incorporated into the organic binder.

Preferably, there is a step of additionally causing at least some of the pore-forming agent to align along a common axis. This may be achieved, for example, by placing the body containing the pore-forming agent into an elongated mould with space to expand at the ends of the mould. The pore-forming agent, such as yeast, is allowed to produce the pores within the confines of the sides of mould, thus forcing the body to elongate along the length of the mould. Alternatively, the pore-forming agent may be aligned simply be extruding the body. This is also described in WO 02/11781.

The ceramic particles are preferably as defined for the first aspect of the invention.

The process preferably comprises a step of additionally incorporating a biologically or pharmaceutically active compound into or onto the fused biomaterial. These compounds are preferably as defined for the first aspect of the invention. They may simply be incorporated by soaking the fused body into a suitable solution containing the biologically or pharmaceutically active compound, prior to drying the product. This allows, for example, the active compound to diffuse within the micropores, midi-pores and macropores of the product.

The invention also includes within its scope biological material obtainable by the process of the invention. Bone implants, dental implants, ear, nose and throat implants comprising the biomaterial, or indeed other implants, are also included within the scope of the invention. The use of the biomaterial as a bone replacement, tooth implant or maxillofacial repair material is also included within the invention. Methods of inducing bone formation in a mammal by implanting a biomaterial according to the invention into a mammal in a manner to induce bone formation on and/or within the biomaterial, are also provided by the invention.

The biomaterial of the invention has been found to have improved bio-compatibility and promotes bone in-growth and cell attachment.

A third object of the invention is to provide a method of repairing or augmenting bone comprising use of a device described above or use of a device produced using any of the methods described above.

A fourth object of the invention is to provide a device suitable for use in a method of repairing or augmenting bone. Preferably the device is as described above and/or produced using any of the methods described above.

The inventor has also realised that using reinforcing metal within the pores of porous material may be used to produce bone repair materials with improved strength.

A further aspect of the invention provides a bone repair or augmentation device comprising a porous body and a reinforcing metal within the pores of at least a portion of pores making the porous body.

Preferably, substantially all of the device comprises the reinforcing metal. The metal is preferably present within the elongated macropores and/or midipores, where present. The reinforcing material may also be present within the micropores of the porous body, for example, if the viscosity of the metal, when heated, is low enough to move into the micropores.

Methods of making a bone repair or augmentation device comprising mixing a porous material with a reinforcing metal, heating to melt the reinforcing metal and cooling the reinforced porous body, are also provided.

Preferably the materials and methods for making the device, such as the porous material and reinforcing metal, are as defined above.

The bone repair or augmentation devices preferably include rods, plates and screws made of the material. These may be made by moulding prior to heating the metal and porous material or by machining the reinforced porous body.

A preferred embodiment of the bone repair or augmentation device of the present invention will now be described by way of example only and with reference to the following drawings in which:—

FIG. 1 is a schematic representation of a top view of a porous body.

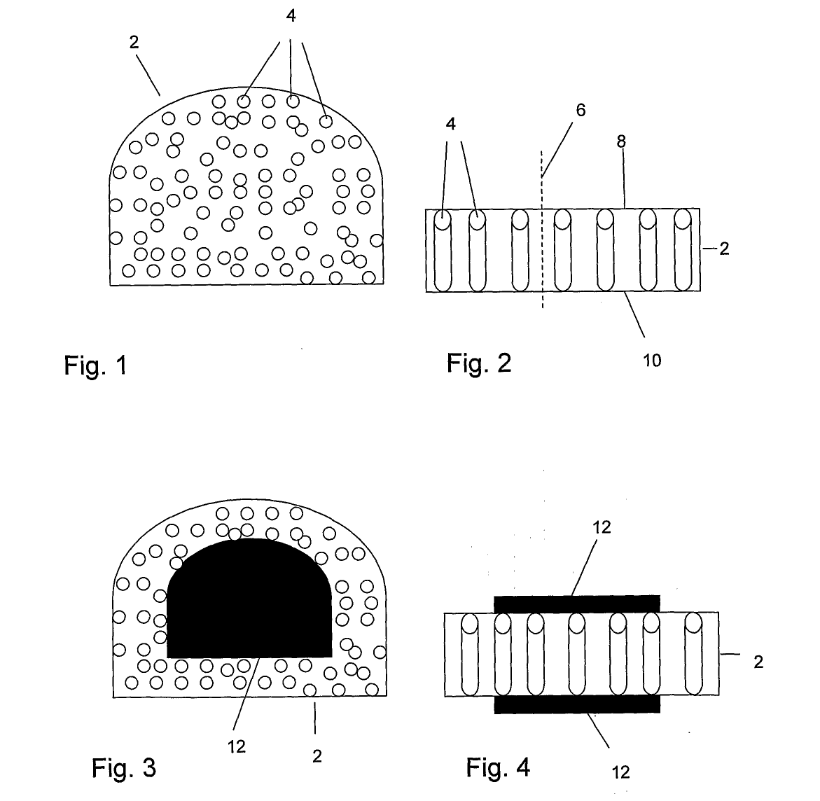

FIG. 2 is a schematic representation of a vertical cross section of the porous body of FIG. 1.

FIG. 3 is a schematic representation of the top view of the porous body of FIG. 1. wherein a part of the porous body is covered with a mask.

FIG. 4 is a schematic representation of a vertical cross section of the porous body of FIG. 3.

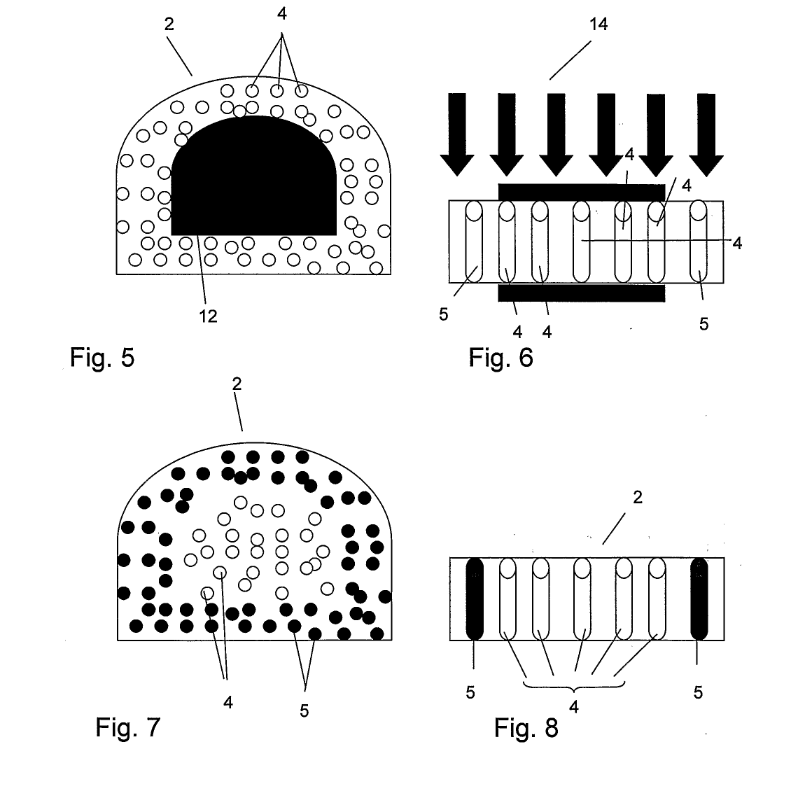

FIG. 5 is a schematic representation of the top view of the porous body of FIG. 3 prior to filling pores with polymer.

FIG. 6 is a schematic representation of a vertical cross section of the porous body of FIG. 5 prior to filling pores with polymer.

FIG. 7 is a schematic representation of a top view of the porous body of FIG. 6 following filling of the pores with polymer and removal of the mask.

FIG. 8 is a schematic representation of a vertical cross section of the porous body of FIG. 7 following filling of the pores with polymer and removal of the mask.

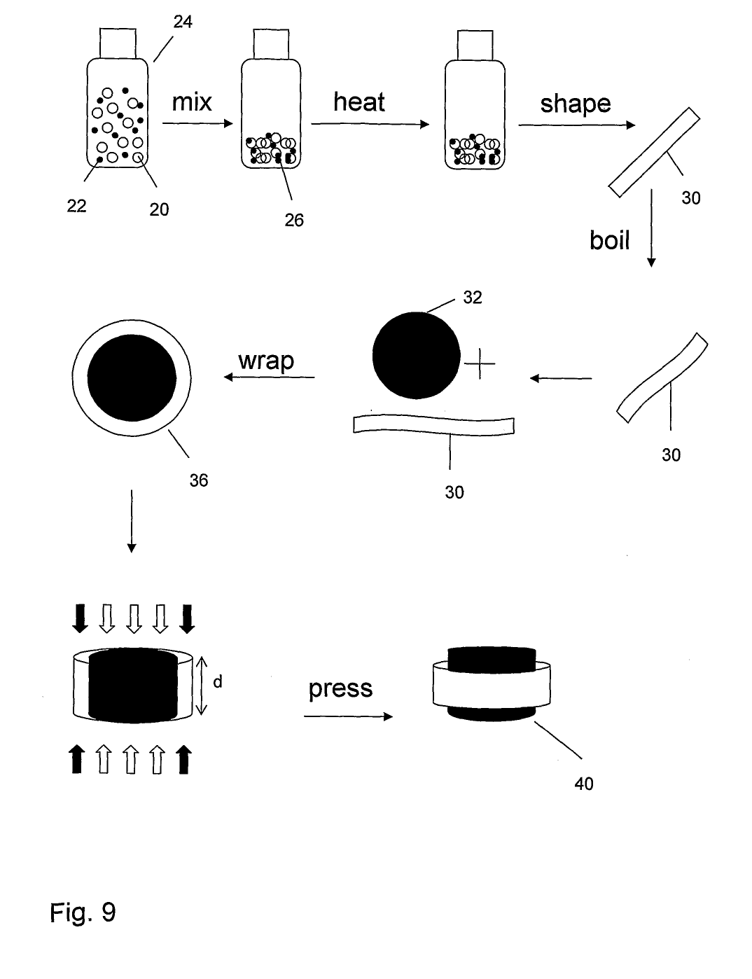

FIG. 9 is a schematic representation of a method for making a device.

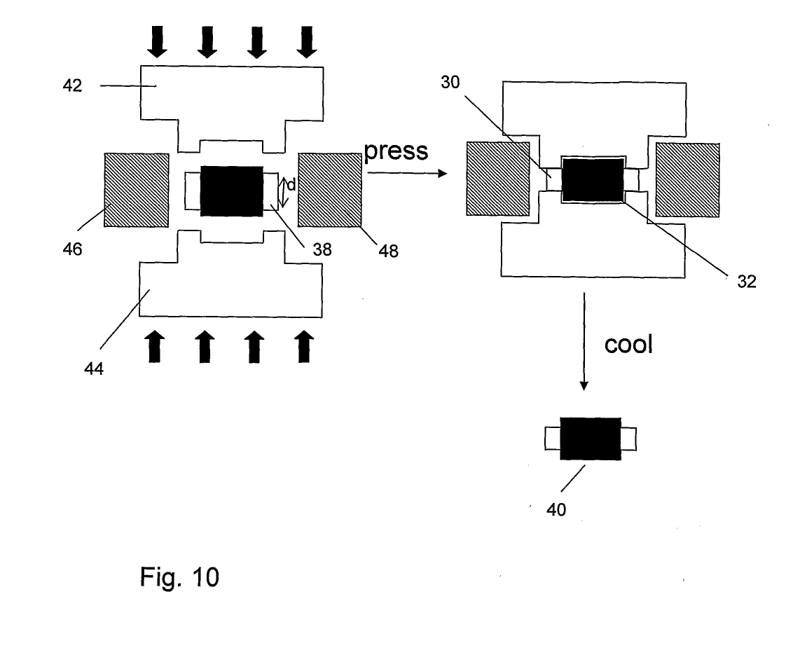

FIG. 10 is a schematic representation of a method for pressing the device produced by the method of FIG. 9.

FIG. 1 is a top view of a porous body 2 for use in the present invention. The pores 4 are distributed substantially evenly throughout the body. The pores 4 may be any suitable combination of macro, midi and mini pores.

FIG. 2 is a schematic representation of a vertical cross section of the porous body of FIG. 1. The pores 4 are aligned substantially along an axis 6 running from the top surface 8 to the bottom surface 10 of the porous body 2. The pores may extend from the top surface 8 to the bottom surface 10. Alternatively, the two or more pores may interconnect to form a substantially continuous pore extending from the top surface 8 to the bottom surface 10.

FIG. 3 is a schematic representation of the top view of the porous body 2 of FIG. 1. wherein a part of the porous body 2 is covered with a mask 12. FIG. 4 is a schematic representation of a vertical cross section of the porous body of FIG. 3. The mask 12 shown in FIGS. 3 and 4 blocks off some of the pores.

FIG. 5 is a schematic representation of the top view of the porous body 2 of FIG. 3 prior to filling pores 4 with polymer. The mask 12 blocks off some of the pores.

FIG. 6 is a schematic representation of a vertical cross section of the porous body 2 of FIG. 5 prior to filling pores 4 with polymer. The arrows 14 represent polymer prior to filling the pores 4. The mask 12 prevents polymer from entering the pores 4 immediately below the mask. The mask does not prevent polymer from entering those pores 5 not shielded by the mask 12.

FIG. 7 is a schematic representation of a top view of the porous body 2 of FIG. 6 following filling of the pores 5 with polymer and removal of the mask 12. FIG. 8 is a schematic representation of a vertical cross section of the porous body of FIG. 7 following filling of the pores with polymer and removal of the mask. In both FIGS. 7 and 8 it is clear that the pores 5 located towards the perimeter of the porous body 2 are filled with polymer whereas the pores located towards the centre of the porous body 2 (i.e. those which were shielded by the mask) are not filled with polymer.

The porous body may be made according to the methods shown in WO 02/11781 or the improved method shown in WO 04/101013, or a combination thereof, incorporated herein, in their entirety.

Briefly, typical ceramic particles such as hydroxyapatite and α- or β-tricalcium phosphate, are mixed with a coating agent such as a starch (especially tapioca starch). Liquid may be added and the mixture steamed for typically 20-30 minutes to form a dough. Typical amounts of material are:

| | | |
|---|---|---|
| Hydroxyapatite | 45.5 | g |
| Water | 38 | ml |
| Optional dispersing agent | 1 | ml |
| Tapioca starch | 9 | g |

A mixture of wheat gluten (13 g) and white strong flour with a high gluten content (15 g), yeast enhancer (vital wheat gluten, diastatic malt, ascorbic acid) and yeast (e.g. Saccharomyces cerevisiae) and, optionally, sugar are mixed with the dough.

The mixture is typically placed in an elongated mould and allowed to prove. The generation of carbon dioxide by the yeast causes the mixture to expand along the length of the elongated mould to align the macropores in the material substantially along the axis of the mould.

This is then set by heating to 100° C. for 20-25 minutes, cut, if desired, into shape, and fired at ca 1350° C. for hydroxyapatite and approx. 1200° C. for tricalcium phosphate.

FIG. 9 is a schematic representation of a method for making a device.

Beads (20) of PCL, a biodegradable polymer, and granules (22) of Tripore, a ceramic, are placed in a 50:50 (by weight) ratio in a glass container (24) and mixed vigorously. The resultant mixture (26) is heated to above the softening point of the PCL in a steam oven (28). The mixture is then shaped into a rod (30). The rod (30) is then further heated by immersing it in boiling water (34). The heated rod (30) is then wrapped around a body of ceramic material (36) to form a composite body (38). The composite body (38) is then pressed through its depth (d) to form a device (40). The device is allowed to cool. Following cooling the device is sterilised by radiation and packaged in a sterile pack. Subsequently, the sterile device may be used in a surgical procedure.

FIG. 10 is a schematic representation of a method for pressing the composite body (38) produced by the method of FIG. 9.

The composite body (38) is placed in a 2-part press (42, 44). The press is used to compress (denoted by black arrows) the composite body (38) throughout its depth (d). The press (42, 44) is designed so that the perimeter of the composite body (30) is compressed but the internal ceramic body (32) is not compressed. This prevents the risk of the ceramic body shattering during the pressing. The resultant device (40) is released from the press and allowed to cool prior to being used in a surgical procedure.

Mould sides (46, 48) are preferably provided to prevent material escaping to the sides of the press (42, 44) on pressing the composite body (38).

1. A bone repair or augmentation device comprising a porous body, the porous body comprising pores characterised in that a portion of the porous body additionally comprises a reinforcing agent within the pores of that portion of the porous body.

2. A device according to claim 1, comprising a plurality of interconnecting macropores which are substantially aligned along an axis.

3. A device according to claim 1 or 2 comprising an upper surface and a lower surface and one or more outer edges connecting the upper surface to the lower surface, wherein more reinforcing agent is located in pores towards the outer edges then in pores located in the centre of the device.

4. A device according to claim 3, wherein the plurality of interconnecting pores align substantially along an axis running through the device from the upper surface to the lower surface.

5. A device according to any preceding claim wherein the porous body comprises a biomaterial having a plurality of connecting micropores of an average diameter of between 1 μm and 10 μm substantially evenly distributed through the biomaterial.

6. A device according to claim 5, composed of a plurality of particles, each particle being partially fused to one or more adjacent particles to form a lattice defining said micropores.

7. A device according to any of claim 5 or 6, wherein each particle has an average diameter of 1 μm to 10 μm.

8. A device according to any of claims 5 to 7 additionally comprising a plurality of elongated macropores having an average diameter of between 150 μm and 500 μm.

9. A device according to any of claims 5 to 8 additionally comprising a plurality of substantially spherical midipores having an average diameter of between 50 μm and 150 μm.

10. A device according to any of claims 5 to 9 wherein the biomaterial is selected from the group comprising (i) ceramic and (ii) bioactive glass.

11. A device according to claim 10 wherein the ceramic comprises at least one type of calcium phosphate.

12. A device according to claim 11 wherein the calcium phosphate is α- or β-tricalcium phosphate or hydroxyapatite or a mixture thereof.

13. A device according to claim 10 wherein the bioactive glass comprises a controlled release glass.

14. A device according to any preceding claim wherein the reinforcing agent is selected from the group comprising polymers and metals.

15. The device according to claim 14 wherein the polymer is a thermoplastic.

16. The device according to claim 15 wherein the polymer is selected from the group comprising biodegradable polyesters.

17. The device according to claim 16 wherein the metal is selected from the group comprising magnesium and magnesium alloys.

18. A device according to any preceding claim wherein the portion not comprising reinforcing agent comprises one or more biologically or pharmaceutically active compounds.

19. A device according to claim 18 wherein the pharmaceutically or biologically active compound is selected from the group comprising stem cells, growth factors, bone morphogenetic protein, osteogenic protein, an enzyme, a vitamin, a trace mineral.

20. A device according to any preceding claim further comprising at least one aperture through which a surgical screw may be inserted.

21. A device according to any preceding claim wherein the device is for at least partial replacement of an intervertebral disc.

22. A method for making a bone repair or augmentation device, the device having a perimeter and an internal region comprising:
(a) mixing a biodegradable reinforcing agent with a biomaterial selected from the group comprising ceramic materials and bioactive glasses to form a mixture;
(b) heating the mixture to above the softening point of the reinforcing agent;
(c) moulding the mixture around at least a portion of the internal region.

23. A method according to claim 22 wherein step (c) comprises forming the product of step (b) into an elongate shape.

24. A method according to claim 23 wherein step (c) comprises wrapping the elongate shape around the internal region to form at least a portion of the perimeter.

25. A method according to claim 24 wherein prior to the wrapping the elongate shape is further softened.

26. A method according to any of claims 22 to 25 wherein the elongate shape has a thickness of between 1 mm and 10 mm.

27. A method according to claim 22 wherein step (c) comprises:
(i) exposing the mixture of step (a) to high pressure to form a loosely packed body; and
(ii) locating at least a portion of the internal region adjacent the compact body.

28. A method according to claim 27 wherein at least a portion of the internal region is inserted within a perimeter defined by the loosely packed body.

29. A method according to any of claims 22 to 28 further comprising fusing the reinforcing agent.

30. A method according to claim 29 wherein the fusing is carried out in a furnace.

31. A method according to claim 29 or 30 wherein the fusing at least partially adheres the internal region to the loosely packed body.

32. A method according to any of claims 22 to 31 further comprising pressing the device.

33. A method according to claim 32 wherein the pressing compresses the perimeter to a greater extent than it compresses the internal region.

34. A method for making a bone repair or augmentation device, the device having a depth, a width, a perimeter and an internal region, the method comprising:
(i) forming a porous body, the porous body comprising a ceramic material having a plurality of pores
(ii) placing a mask on at least the upper surface of the porous body to cover some of the pores and to leave some of the pores exposed
(iii) at least partially filling the exposed pores with a reinforcing agent and
(iv) removing the mask from at least the upper surface of the porous body to expose pores located under the mask.

35. The method according to claim 34 further comprising, at the same time as or after step (ii), placing a mask on the lower surface of the porous body to cover some of the pores and to leave some of the pores exposed and at the same time as or after step (iv) removing the mask from the lower surface of the porous body to expose pores located under the mask.

36. The method according to any of claims 34 to 35 wherein the at least partial filling of the exposed pores is by immersing the porous body in a reinforcing agent and/or by injecting a reinforcing agent into the pores.

37. The method according to claim 36 wherein the injection is carried out by injection moulding.

38. The method according to claim 36 or 37 wherein during filling the reinforcing agent is in a fluid or molten state.

39. A method according to any of claims 22 to 38 wherein the internal region comprises a ceramic material.

40. A method according to any of claims 22 to 39 further comprising allowing the product to cool.

41. A method according to any of claims 22 to 40 wherein at least one of the reinforcing agent, ceramic material or internal region is porous.

42. A method according to any of claims 22 to 41 wherein the reinforcing agent is selected from the group comprising polymers and metals.

43. A method according to claim 42 wherein the polymer is a thermoplastic.

44. A method according to claim 43 wherein the polymer is selected from the group comprising biodegradable polyesters.

45. A method according to any claim 44 wherein the polymer is PCL.

46. A method according to any of claims 42 to 45 wherein the polymer is provided in bead form.

47. A method according to claim 42 wherein the metal is selected from the group comprising magnesium and magnesium alloys.

48. A method according to any of claims 22 to 47 wherein the internal region comprises one or more biologically or pharmaceutically active compounds.

49. A method according to claim 48 wherein the pharmaceutically or biologically active compound is selected from the group comprising stem cells, growth factors, bone morphogenetic protein, osteogenic protein, an enzyme, a vitamin, a trace mineral.

50. A method according to any of claims 22 to 49 further comprising forming at least one aperture through which a surgical screw may be inserted.

51. The method according to any of claims 22 to 50 wherein the product of the method comprises a device according to any of claims 1 to 21.

52. A method of repairing or augmenting bone comprising using a device according to any of claims 1 to 19.

53. A device according to any of claims 1 to 19 for use in a method of repairing or augmenting bone.

54. A bone repair or augmentation device comprising a porous body and a reinforcing metal within at least a portion of pores making the porous body.

55. A device according to claim 54, wherein the porous body comprises a ceramic or a bioactive glass.

56. A device according to claim 54 or claim 55, wherein the metal is magnesium or a magnesium alloy.

57. A bone repair or augmentation device substantially as described herein with reference to the accompanying drawings.

58. A method for making a bone repair or augmentation device substantially as described herein with reference to the accompanying drawings.
